(19) United States
(12) Patent Application Publication
(10) Pub. No.: US 2026/0284400 A1
(43) Pub. Date: Sep. 24, 2026

(54) ENHANCED ADAPTIVE BRAIN STIMULATION SYSTEM AND METHOD

(71) Applicant: IMRAN KHURSHID ALAM, Plano, TX (US)

(72) Inventor: IMRAN KHURSHID ALAM, Plano, TX (US)

(21) Appl. No.: 19/086,204

(22) Filed: Mar. 21, 2025

(51) Int. Cl. A61N 1/36; A61B 5/369; A61B 5/389; A61N 1/05; G16H 10/60

(52) U.S. Cl. CPC A61N 1/36135; A61B 5/369; A61B 5/389; A61N 1/0526; G16H 10/60

(57) ABSTRACT

The present disclosure provides a brain stimulation system comprising an implantable electrode assembly configured to interface with neural tissue, an artificial intelligence (AI) powered controller operatively connected to the electrode assembly and configured to dynamically adjust stimulation parameters based on real-time neurophysiological feedback, and a wireless interface configured to enable remote monitoring and programming of the stimulation parameters. The electrode assembly includes a flexible polymer substrate with multiple electrode contacts arranged in a radial pattern. The AI-powered controller utilizes a machine learning model trained on patient-specific neurological data to personalize stimulation therapy. The system further includes a cloud-based data analytics system for analyzing aggregated patient data and providing predictive analytics for therapy optimization.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The present disclosure relates to brain stimulation techniques, and more particularly to an AI-enhanced adaptive brain stimulation system and method for treating neurological and psychiatric disorders using real-time biofeedback and cloud-based data analytics.

### BACKGROUND

Brain stimulation therapies have emerged as promising approaches for treating various neurological and psychiatric disorders. These techniques involve directly modulating neural activity through electrical or magnetic stimulation of specific brain regions. Common brain stimulation methods include deep brain stimulation (DBS), transcranial magnetic stimulation (TMS), and transcranial direct current stimulation (tDCS).

While brain stimulation has shown efficacy for conditions like Parkinson's disease, epilepsy, and depression, current approaches have limitations. Many existing systems use fixed stimulation parameters that do not adapt to the patient's changing brain state or symptoms over time. Additionally, stimulation protocols are typically determined based on population-level data rather than being personalized for each individual patient.

There is growing interest in developing closed-loop brain stimulation systems that can dynamically adjust stimulation parameters based on real-time physiological feedback. Such adaptive stimulation could potentially improve therapeutic outcomes while minimizing side effects. However, implementing closed-loop control for brain stimulation presents technical challenges.

Recent advances in artificial intelligence, particularly machine learning algorithms, have created new opportunities for enhancing brain stimulation therapies. AI techniques could potentially be leveraged to process complex neural data streams in real-time and make intelligent decisions about stimulation parameters. Cloud computing infrastructure also enables aggregation and analysis of large-scale patient data to inform stimulation protocols.

Integrating AI capabilities with brain stimulation hardware could lead to more personalized and effective neuromodulation therapies. However, realizing the potential of AI-enhanced adaptive brain stimulation requires overcoming hurdles related to on-device processing, wireless communication, power consumption, and clinical validation. Addressing these challenges could advance the field of therapeutic brain stimulation

### BRIEF SUMMARY OF THE INVENTION

The instant invention in one form is directed to a brain stimulation system comprising an implantable electrode assembly configured to interface with neural tissue, an artificial intelligence (AI) powered controller operatively connected to the electrode assembly and configured to dynamically adjust stimulation parameters based on real-time neurophysiological feedback, and a wireless interface configured to enable remote monitoring and programming of the stimulation parameters. The electrode assembly includes a flexible polymer substrate with multiple electrode contacts arranged in a radial pattern. The AI-powered controller utilizes a machine learning model trained on patient-specific neurological data to personalize stimulation therapy. The system further includes a cloud-based data analytics system for analyzing aggregated patient data and providing predictive analytics for therapy optimization.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a brain stimulation system is provided. The system includes an implantable AI-enhanced electrode assembly, a real-time adaptive neuromodulation controller, and a wireless interface for monitoring and programming.

According to other aspects of the present disclosure, the system may include one or more of the following features. The system may include an AI-powered algorithm for dynamic adjustment of neuromodulation parameters based on real-time EEG and EMG feedback. The system may include a cloud-based data analytics system that predicts neurological conditions and optimizes stimulation therapy. The system may use biofeedback-responsive closed-loop control to enhance stimulation efficiency and minimize side effects. The system may include a wireless remote-control system for external modulation of stimulation parameters, allowing real-time physician intervention. The system may include an implantable power system utilizing inductive charging and energy harvesting to sustain long-term functionality. The system may include a smart electrode array with nano-coatings to enhance biocompatibility and reduce inflammatory responses. The system may include a real-time AI-based seizure detection algorithm integrated within the implantable system. The system may include a dual-mode stimulation protocol allowing automatic switching between electrical and magnetic stimulation. The system may include a self-learning neural stimulation algorithm adapting to patient responses over time.

According to another aspect of the present disclosure, a method of brain stimulation therapy is provided. The method includes an AI-driven algorithm autonomously modifying neuromodulation parameters in response to electroencephalographic (EEG) or electromyographic (EMG) biofeedback to optimize therapeutic efficacy.

According to yet another aspect of the present disclosure, a wearable or implantable wireless neurostimulation device is provided. The device is equipped with a cloud-based data analytics system for remote patient monitoring, predictive analytics, and physician-controlled parameter adjustments in treating neurological disorders.

The foregoing general description of the illustrative embodiments and the following detailed description thereof are merely exemplary aspects of the teachings of this disclosure and are not restrictive.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which reference numerals are used to refer to similar components. When reference is made to a reference numeral without specification to an existing sub-label, it is intended to refer to all such multiple similar components.

Non-limiting and non-exhaustive examples are described with reference to the following figures.

FIG. 1 illustrates a perspective view of a brain stimulation device, according to aspects of the present disclosure.

FIG. 2 depicts a sectional view of the brain stimulation device mounted to skull bone, according to an embodiment.

FIG. 3 shows various types of electrodes for connecting to the brain stimulation device, according to aspects of the present disclosure.

FIG. 4 illustrates a perspective view of a remote-control unit for the brain stimulation device, according to an embodiment.

FIG. 5 depicts a perspective view of the brain stimulation device compared to brain tissue, according to aspects of the present disclosure.

FIG. 6 shows a perspective view of an electrode assembly for the brain stimulation device, according to an embodiment.

FIG. 7 illustrates a perspective view of a single electrode attached to the brain stimulation device, according to aspects of the present disclosure.

FIG. 8 depicts a perspective view of a smartphone device displaying a medical interface, according to an embodiment.

FIG. 9 shows a perspective view of a strip electrode for brain stimulation applications, according to aspects of the present disclosure.

FIG. 10 illustrates a detailed perspective view of a brain stimulation electrode assembly, according to an embodiment.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The examples described are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to an AI-enhanced adaptive brain stimulation system and method. This system integrates advanced artificial intelligence algorithms with neuromodulation technology to provide targeted and adaptive stimulation of neural circuits. The system may be designed to address a broad spectrum of neurological and psychiatric disorders.

In some cases, the system may incorporate real-time analysis and adaptation of stimulation parameters based on neurophysiological feedback. The system may utilize a smart electrode array with micro-sensors capable of autonomously adjusting current intensity and frequency.

The system may include cloud-based AI algorithms for predictive analytics and prevention of various neurological events. A wireless interface may be incorporated for remote monitoring and programming of stimulation parameters.

In some implementations, the system may improve upon prior skull-implanted electrode assemblies by integrating biofeedback-responsive stimulation protocols. The system may employ machine learning models trained on patient-specific neurological data to personalize stimulation therapy.

The adaptive AI algorithms may enable automatic adjustments to stimulation parameters such as frequency, pulse width, and amplitude. Neural activity prediction models may be utilized to prevent overstimulation and reduce adverse effects.

In some cases, the system may integrate biofeedback sensors for continuous monitoring of brain function. A closed-loop control mechanism may modify stimulation based on brainwave activity to ensure optimal neuromodulation response.

The system and method described herein may represent an advancement in brain stimulation technology by merging AI capabilities with precision neuromodulation techniques. This approach may offer potential improvements in treating various neurological and psychiatric conditions.

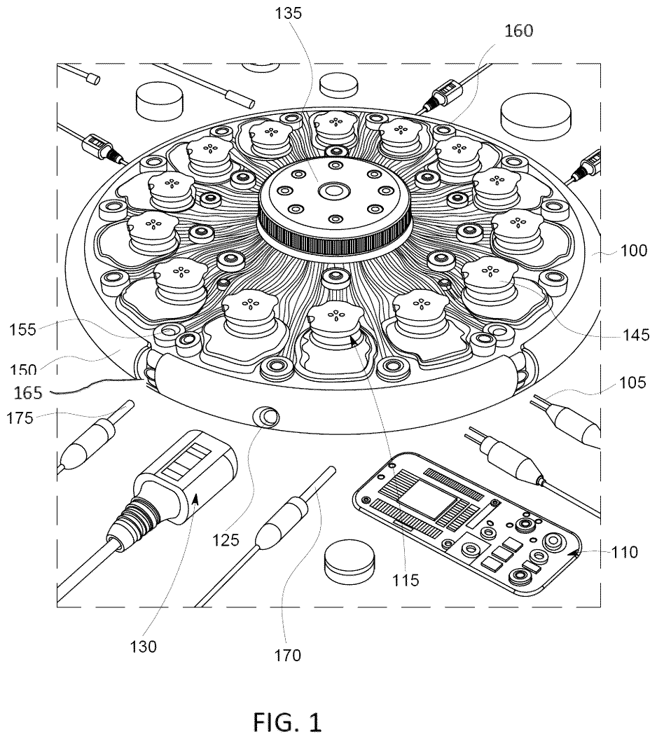

The implantable electrode assembly may be a key component of the brain stimulation device 100. In some cases, the brain stimulation device 100 may incorporate a linear electrode array 105 as shown in FIG. 1. The linear electrode array 105 may be positioned around the periphery of the circular structure of the brain stimulation device 100.

The brain stimulation device 100 may include multiple electrode contract modules 155 arranged in a circular pattern to enable targeted neural stimulation.

As shown in FIG. 1, the electrode assembly may be arranged in a circular array with multiple stimulation modules surrounding a central hub. The modules may contain biofeedback sensors and neuromodulation components with metallic contacts for interfacing with neural tissue. The Electric Brain Stimulation device 100 showcasing its advanced design with AI-powered neuromodulation, biofeedback sensors, and wireless capabilities. The Electric Brain Stimulation device 100 is comprised of linear arrays electrodes 105, wireless remote interface circuit board 110, stimulators 115, AI sensors 125, connector 130, LED Status Indicator 135, Central Processing unit 140, Neural interface connectors 145, Protective outer shell 150, Electrode contract modules 155, Microelectrode array 160, Diagnostic and Programming Interface 165, Precision Surgical Instruments 170 and Wireless Power coil 175.

This AI-powered electrical brain stimulator represents a major advancement in neuromodulation technology. It offers:
a. Precision-targeted stimulation.
b. Adaptive therapy based on real-time neural signals.
c. Minimal side effects.
d. Wireless remote monitoring & AI-driven automation.
e. Predictive analytics for neurological disorders.

Electric Brain Stimulation device 100 comprises of the following:
a. Central Processing Unit (Middle Circular Component)
   i. Function: The core computational unit of the device, responsible for real-time data processing, AI-driven neural signal modulation, and device control.
   ii. Materials: Likely contains silicon-based microprocessors and specialized AI chips to adjust stimulation patterns dynamically.
b. Electrode Contact Modules (Multiple Small Metallic Components Around the Periphery)
   i. Function: These modules deliver electrical stimulation to the brain and record neural activity.
   ii. Material: Typically made of platinum-iridium for biocompatibility, corrosion resistance, and high conductivity.
c. Microelectrode Array (Fine Circuitry Spread Across the Surface)
   i. Function: Collects detailed neural signals from multiple regions to provide precise stimulation and feedback.
   ii. Material: Flexible microelectronics based on polyimide or graphene-based conductive films.
d. Wireless Power Coil (Encased in the Outer Circular Structure)
   i. Function: Enables inductive charging, removing the need for direct power connections.
   ii. Material: Typically made from copper or silver-coated wire wrapped in a biocompatible polymer.
e. Neural Interface Connectors (Located at the Base of the Electrodes)
   i. Function: Facilitate the secure attachment of electrode leads to ensure efficient signal transfer.
   ii. Material: Gold-plated or platinum-iridium alloy to optimize signal conductivity.
f. LED Status Indicators (Small Blue Lights Around the Device)
   i. Function: Provide real-time status updates about the device's operation, stimulation status, and connectivity.
g. Protective Outer Shell (Circular Metal Frame with Fasteners)
   i. Function: Protects internal electronics while allowing heat dissipation.
   ii. Material: Titanium or stainless steel, chosen for biocompatibility, strength, and MRI compatibility.
h. Diagnostic and Programming Interface (External Circuit Board in the Image)
   i. Function: Allows doctors and researchers to program, diagnose, and adjust stimulation parameters remotely.
i. Precision Surgical Instruments (Displayed Around the Device)
j. Function: Tools used during implantation and maintenance, including neural probes, calibration tools, and micro-adjustment screwdrivers.

Overall Functionality:
a. The device records brain activity, applies targeted stimulation, and adapts therapy dynamically based on real-time AI analysis.
b. It supports wireless power transfer, eliminating the need for frequent battery replacements.
c. The outer shell ensures implant stability and long-term durability.

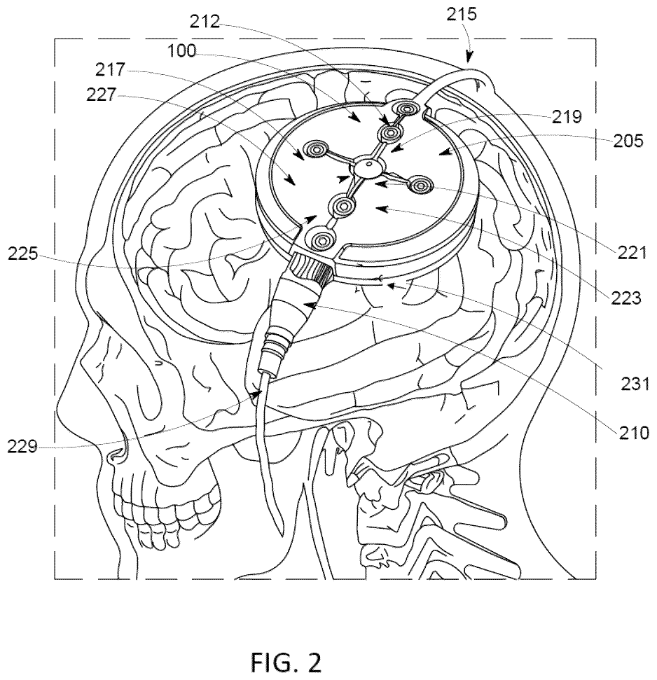

As illustrated in FIG. 2, an electrode lead 210 may extend from the housing of the brain stimulation device 100 to make contact with brain tissue. The electrode lead 210 may terminate in contact platinum iridium electrodes 231 that interface directly with neural tissue.

FIG. 2 shows the housing 205, flexible electrode lead 210, Cap holding 212, Grounding 215, AI powered stimulation control unit 217, implantable electrode housing 219, Fixation points 221, Wireless power and data interface 223, Biocompatible surface coating 225, Nural Signal Processor 227, Electrode leads 229, Platinum Iridium Electrodes 231.

FIG. 2 shows the Electric Brain Stimulation device 217 securely attached to the skull bone, with a flexible electrode lead resting on the surface of the brain.

The Brain Stimulation Device 100 comprises:
1. Implantable Electrode Housing (Metallic Circular Shell)
   a. Location: The main external structure of the device, mounted on the skull.
   b. Function: Provides a protective casing for internal components, ensuring durability and biocompatibility. It is typically made of titanium or medical-grade stainless steel for corrosion resistance and MRI compatibility.
2. Fixation Points (Screw-Like Structures on the Shell)
   a. Location: Four equidistant points securing the device to the skull.
   b. Function: These self-locking fixation points anchor the implant, preventing movement. They may be biodegradable or permanent, depending on surgical needs.
3. Electrode Leads (Cables Extending from the Device into the Brain)
   a. Location: Thin cables running from the device into the brain's surface.
   b. Function: These leads deliver electrical stimulation and record neural signals. They are made of flexible polymer materials to minimize mechanical stress on the brain.
4. Platinum-Iridium Electrodes (Micro-Contacts at the Tip of the Leads)
   a. Location: The ends of the electrode leads inside the brain tissue.
   b. Function: These high-precision electrodes conduct stimulation while ensuring high fidelity in neural recording. Platinum-iridium is used for durability, conductivity, and biocompatibility.
5. AI-Powered Stimulation Control Unit (Internal Processing System in the Device)
   a. Location: Inside the metallic housing.
   b. Function: Uses real-time AI algorithms to adjust stimulation patterns, optimizing therapy based on neural activity. The AI ensures adaptive neuromodulation based on patient-specific needs.
6. Power Supply & Wireless Charging Coil (Internal Energy Management System)
   a. Location: Inside the implant casing, near the outer surface.
   b. Function: Uses inductive charging or energy harvesting to power the device wirelessly, eliminating the need for battery replacement.
7. Neural Signal Processor (Integrated Microchip for Data Processing)
   a. Location: Embedded within the implant.
   b. Function: Captures, filters, and interprets brain wave data before sending it to the AI unit for real-time adjustments.
8. Biocompatible Surface Coating (Covering the Metal Shell & Electrodes)
   a. Location: Coats the outer shell and electrode contact surfaces.
   b. Function: Reduces immune response, prevents inflammation, and improves long-term device acceptance. Materials like Parylene-C or hydrophilic coatings may be used.
9. Communication Interface (wireless Data Transmission Module)
   a. Location: Integrated within the casing.
   b. Function: Enables the wireless connection to external devices, allowing doctors to monitor and adjust settings via a smartphone or computer interface.
10. Skull Integration Mechanism (Interface Between the Implant and the Skull Bone)
   a. Location: Underside of the implant, where it contacts the skull.
   b. Function: Some models feature a porous structure or bone-mimicking material that promotes osseointegration (natural bonding with the skull) for better stability.

### Overall Functionality

a. Primary Purpose: The device modulates neural activity using precise electrical stimulation while recording brain signals for adaptive therapy.
b. Application Areas: Used in treating neurological disorders like epilepsy, Parkinson's, depression, and chronic pain.
c. Advantages: Wireless power, AI-driven adjustments, and real-time monitoring ensure high precision and minimally invasive.

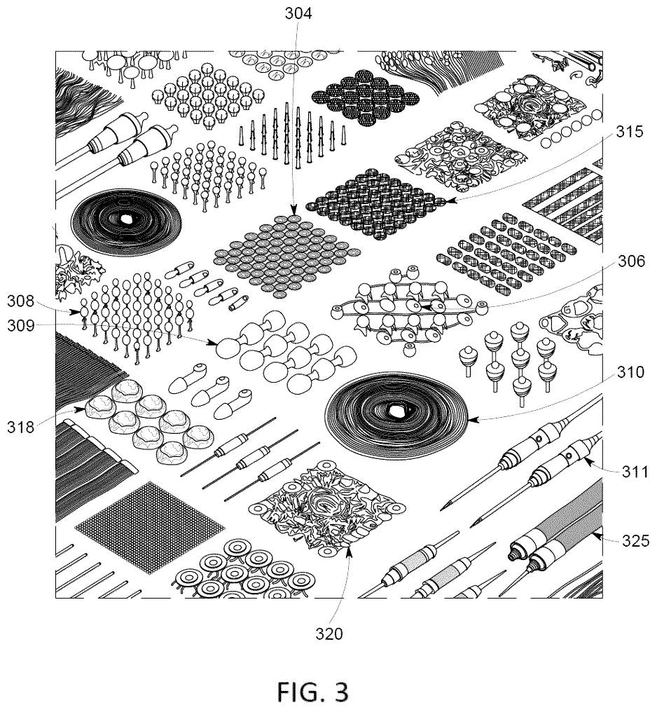

In some cases, the electrode assembly may incorporate various electrode configurations as depicted in FIG. 3. These may include circular arrays, linear arrays, and matrix patterns, allowing for versatile stimulation options. The electrodes may be constructed from materials such as platinum-iridium to optimize conductivity and biocompatibility.

FIG. 3 demonstrates various electrode configurations that may be used with the brain stimulation device 100 shown in FIG. 1. These configurations may include a gamma electrode array 304, a dental electrode array 306, a strip electrode array 308, and a multi-contact array 309. The figure also shows a circular electrode array 310, a deep brain electrode 311, and a matrix electrode array 315. In some implementations, the brain stimulation device 100 shown in FIG. 1 may utilize a hybrid stimulation array 318, a custom electrode mesh 320, or a precision microprobe 325.

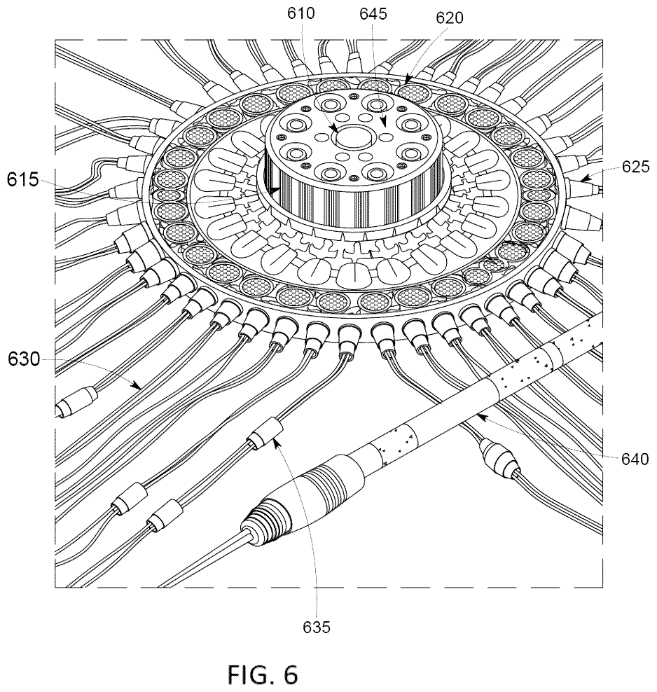

As depicted in FIG. 6, the electrode assembly may feature a central hub 610 surrounded by an electrode array 630 radiating in a symmetrical pattern. The electrode array 630 may provide multiple stimulation points. Contact points 625 may extend outward from the central structure, allowing for interface with neural tissue. A neural interface 635 may be incorporated to enable interaction with brain tissue.

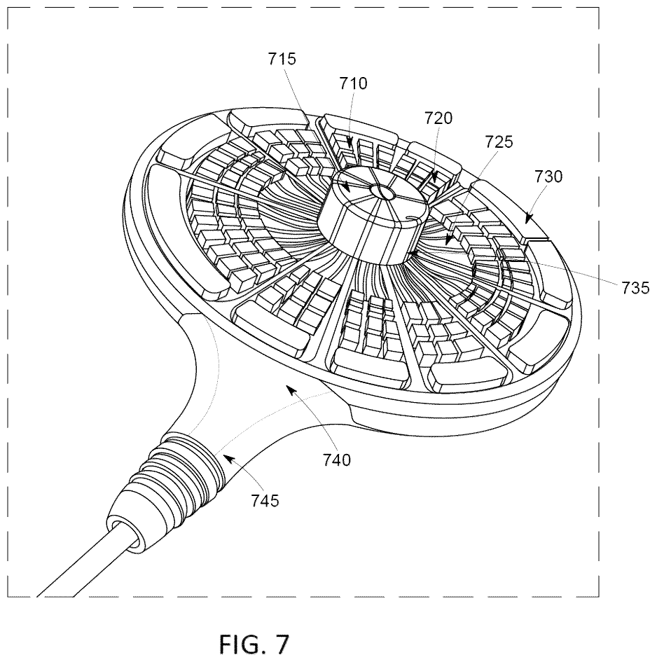

FIG. 7 illustrates a single electrode attached to the brain stimulation device 100. The electrode may include a high-density electrode array 710 arranged in a circular configuration around a central processing hub 715. Contact points 730 may be positioned around the periphery of the device.

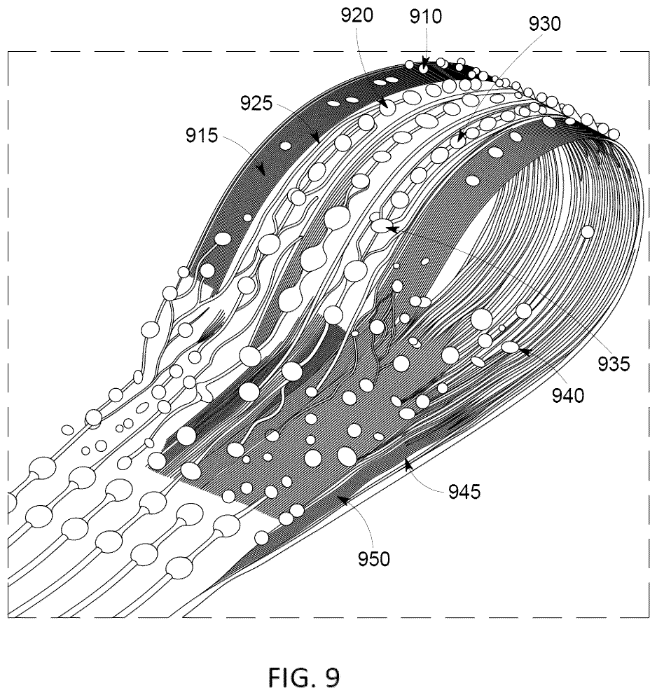

As shown in FIG. 9, the electrode may feature multiple layers, including a flexible polymeric layer and a biocompatible layer. The nano-coatings applied to the surface may enhance conductivity while minimizing tissue reaction. These coatings may be designed to promote cell adhesion, reduce protein adsorption, or release anti-inflammatory agents to mitigate the foreign body response.

In some cases, the electrode assembly may take the form of a strip electrode 910, as shown in FIG. 9. The strip electrode 910 may comprise a polymer substrate 915 that serves as the base structure. Conductive pathways 920 may be integrated into the design, arranged in parallel tracks along the length of the strip electrode 910. A biocompatible coating 925 may be applied to the surface to enhance tissue compatibility. Contact points 930 may be distributed along the length of the strip electrode 910, providing interfaces for electrical stimulation. The strip electrode 910 may include neural interfaces 935 and microelectrode arrays 940. Signal transmission lines 945 may run through the length of the device, facilitating the transfer of electrical signals. Stimulation filaments 950 may extend from the main body of the strip electrode 910, providing additional points of contact for neural stimulation.

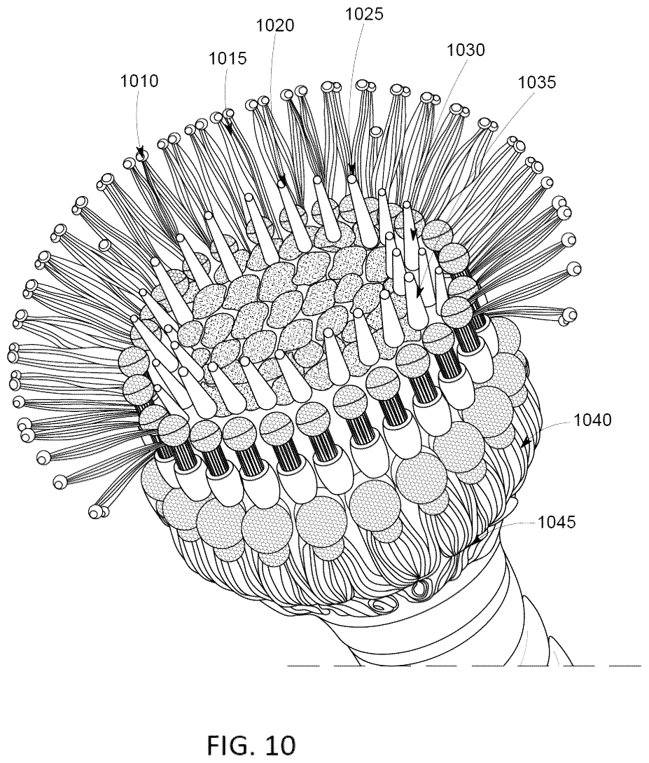

FIG. 10 provides a detailed view of an electrode assembly 1010. The electrode assembly 1010 may feature microfiber filaments 1015 extending radially outward from a polymer base 1020. Contact points 1025 may be distributed across the surface of the structure. Coated electrodes 1030 may be arranged in a circular pattern and may incorporate platinum-iridium materials for conductivity and biocompatibility. A stimulation array 1035 may comprise multiple electrode elements arranged in concentric rings. Neural signal transmission wires 1040 and high-precision microfilaments 1045 may extend from the polymer base 1020 and terminate in spherical tips that serve as individual stimulation sites.

The electrode assembly may incorporate smart electrode arrays with nano-coatings to enhance biocompatibility and reduce inflammatory responses. These nano-coatings may be applied to the contact points 1025, coated electrodes 1030, and precision filaments 1045. The nano-coatings may help minimize tissue reaction and improve long-term stability of the electrode-tissue interface.

### Microfiber Filament Elements (Highlighted in the Image as Thin, Wire-Like Structures)

a. Location: Extending outward from the electrode tip in a radial pattern.
b. Function: These filaments provide high-density neural interfacing, allowing for precise signal transmission and stimulation. The thin structure minimizes tissue damage while maintaining high sensitivity.

### Flexible Biocompatible Polymer Base (Highlighted in the Image as the Blue Underlying Structure)

a. Location: The foundation of the electrode, which holds and supports the microfiber filaments.
b. Function: Made from medical-grade polymers such as PDMS (Polydimethylsiloxane), Parylene, or Polyimide, ensuring flexibility while reducing mechanical stress on brain tissue. It helps prevent immune rejection and maintains structural integrity in long-term implantation.

### Platinum-Iridium Coated Filaments (Highlighted as the Spherical and Wire-Like Conductive Parts in the Image)

a. Location: Found on the tips of the microfiber filaments, forming direct contact points with neurons.
b. Function: Platinum-iridium alloy provides excellent conductivity, corrosion resistance, and long-term stability, ensuring consistent electrical signal transmission for neural modulation.

### Nano-Coatings for Enhanced Biocompatibility (Highlighted as the Textured/Porous Surface on the Electrode Contact Points)

a. Location: Applied to the filaments and electrode surfaces to optimize performance.
b. Function: These coatings, often made from Iridium Oxide (IrOx), PEDOT
c. (Conductive Polymer), or Diamond-Like Carbon (DLC), enhance conductivity, reduce impedance, and prevent biofouling, improving the longevity of the implant.

In some implementations, the nano-coatings may incorporate biomimetic materials that closely resemble the extracellular matrix of neural tissue. This approach may help camouflage the electrode from the immune system, potentially reducing long-term inflammation and scar tissue formation around the implant.

The combination of advanced power systems and biocompatible nano-coatings may contribute to the overall longevity and effectiveness of the brain stimulation device. These features may help address challenges associated with long-term implantation, such as power management and tissue compatibility, potentially improving the stability and durability of the neuromodulation therapy.

The brain stimulation system may incorporate advanced stimulation protocols to enhance therapeutic efficacy and adaptability. These protocols may include dual-mode stimulation capabilities, real-time seizure detection, and self-learning algorithms for personalized treatment.

In some cases, the system may employ a dual-mode stimulation protocol that allows for automatic switching between electrical and magnetic stimulation. This capability may enable the device to utilize different stimulation modalities based on the patient's neural response or specific treatment requirements. The dual-mode protocol may incorporate both electrical current delivery through the electrode contacts and magnetic field generation for non-invasive stimulation.

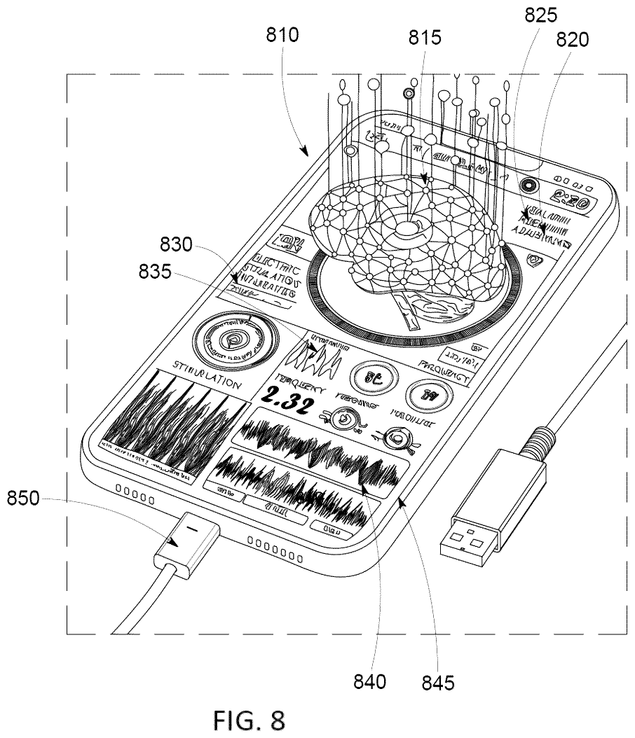

The system may include a real-time AI-based seizure detection algorithm integrated within the implantable device. This algorithm may continuously analyze incoming neural signals to identify patterns or markers indicative of an impending seizure. As shown in FIG. 8, the user interface may display real-time neuromodulation data, including the stimulation frequency and the display time. These parameters may be dynamically adjusted based on the seizure detection algorithm's output.

In some implementations, the seizure detection algorithm may utilize machine learning techniques to improve its accuracy over time. The algorithm may analyze historical data from the patient's neural activity patterns and correlate them with known seizure events to refine its predictive capabilities.

The brain stimulation system may also incorporate a self-learning neural stimulation algorithm that adapts to patient responses over time. This algorithm may continuously monitor the effectiveness of the stimulation parameters and make incremental adjustments to optimize therapeutic outcomes.

The self-learning algorithm may analyze various data inputs, including the stimulation frequency displayed on the user interface in FIG. 8, along with other physiological markers and patient feedback. Based on this analysis, the algorithm may modify stimulation parameters such as frequency, amplitude, or pulse width to achieve the desired therapeutic effect while minimizing side effects.

In some cases, the self-learning algorithm may incorporate reinforcement learning techniques, where positive outcomes (e.g., reduced seizure frequency or improved symptom control) are used to reinforce effective stimulation patterns. This approach may allow the system to gradually tailor the stimulation protocol to the individual patient's unique neural characteristics and response patterns.

The combination of dual-mode stimulation, real-time seizure detection, and self-learning algorithms may create a highly adaptive and personalized neuromodulation system. This advanced approach to stimulation protocols may potentially improve treatment outcomes across a range of neurological disorders by providing more targeted and responsive therapy.

The brain stimulation system may integrate multiple components to provide AI-enhanced adaptive neuromodulation. In some cases, the system may combine the electrode assembly, AI-powered controller, wireless interface, and biofeedback mechanisms to create a comprehensive neuromodulation platform.

As illustrated in FIG. 1, the brain stimulation device may comprise a circular housing containing multiple components arranged in a radial pattern. This configuration may allow for efficient integration of various functional elements, including stimulation modules, biofeedback sensors, and processing units.

The electrode assembly, as shown in FIG. 2, may be positioned in direct contact with brain tissue. In some implementations, the flexible electrode lead extending from the housing may interface with specific neural circuits, enabling targeted stimulation and recording of brain activity.

The AI-powered controller within the device housing may process incoming neural signals and adjust stimulation parameters in real-time. This adaptive capability may allow the system to respond dynamically to changes in brain activity, potentially optimizing therapeutic outcomes.

In some cases, the wireless interface may facilitate remote monitoring and control of the stimulation system. As depicted in FIG. 8, a smartphone or similar device may display real-time neuromodulation data, including stimulation frequency and other relevant parameters. This interface may enable clinicians to assess treatment efficacy and make adjustments as needed.

The biofeedback integration may provide continuous monitoring of brain function through embedded sensors. In some implementations, the closed-loop control mechanism may utilize this data to modify stimulation based on detected brainwave activity, ensuring responsive neuromodulation.

The system may incorporate cloud-based data analytics for predictive modeling and long-term therapy optimization. This feature may allow for aggregation and analysis of patient data, potentially improving treatment protocols over time.

In some cases, the dual-mode stimulation capability may enable the system to switch between electrical and magnetic stimulation modalities. This flexibility may allow for tailored treatment approaches based on individual patient needs and responses.

The real-time seizure detection algorithm may continuously analyze neural activity patterns. When potential seizure activity is detected, the system may automatically adjust stimulation parameters to potentially prevent or mitigate the seizure.

The self-learning neural stimulation algorithm may adapt to patient responses over time. By analyzing historical data and treatment outcomes, the system may refine its stimulation protocols to potentially enhance therapeutic efficacy for each individual patient.

In some implementations, the power management system may utilize inductive charging and energy harvesting techniques to maintain long-term functionality. This approach may reduce the need for invasive procedures to replace power sources.

The integration of these components may create a comprehensive brain stimulation system capable of adaptive, personalized neuromodulation. By combining advanced hardware with sophisticated AI algorithms and real-time data processing, the system may offer potential improvements in treating a range of neurological and psychiatric disorders.

The brain stimulation device 100 may include an interface circuit board 110 that serves as the central processing unit for the AI-powered controller. In some cases, the interface circuit board 110 may be integrated with a stimulation control unit 217 and a signal processor 227, as shown in FIG. 2. These components may work together to enable real-time analysis and adaptation of stimulation parameters based on neurophysiological feedback.

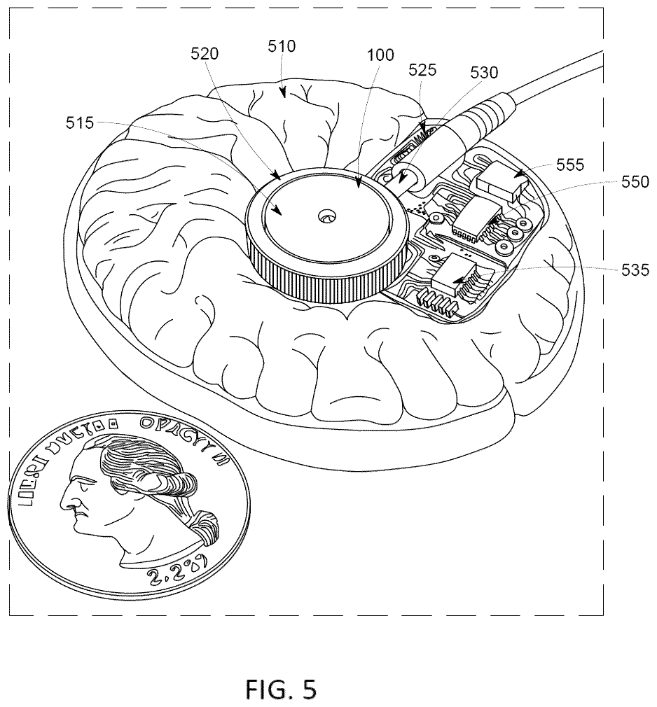

The brain stimulation device 100 may incorporate a processing chip 535, as illustrated in FIG. 5. The processing chip 535 may handle complex computations required for AI-driven algorithms. In some implementations, the processing chip 535 may work in conjunction with a microcontroller 550 to manage various aspects of the device's operation.

FIG. 6 depicts a signal processing unit 645 that may be responsible for processing neural signals and implementing AI-based control algorithms. The signal processing unit 645 may be connected to the electrode array 630, allowing for direct analysis of neural activity data.

In some cases, the brain stimulation device 100 may include a signal modulation unit 735, as shown in FIG. 7. The signal modulation unit 735 may work in tandem with the processing hub 715 to dynamically adjust stimulation parameters based on real-time neural feedback.

The AI-powered controller may incorporate a real-time AI-based seizure detection algorithm integrated within the implantable system. This algorithm may continuously analyze incoming neural signals from the electrode array 630 or the contact electrodes 231 to identify patterns or markers indicative of an impending seizure. The seizure detection algorithm may utilize machine learning techniques to improve accuracy over time, analyzing historical data from the patient's neural activity patterns and correlating them with known seizure events.

The brain stimulation device 100 may also include a self-learning neural stimulation algorithm that adapts to patient responses over time. This algorithm may be implemented within the signal processing unit 645 or the processing chip 535. The self-learning algorithm may continuously monitor the effectiveness of the stimulation parameters and make incremental adjustments to optimize therapeutic outcomes. In some implementations, the algorithm may incorporate reinforcement learning techniques, where positive outcomes (e.g., reduced seizure frequency or improved symptom control) are used to reinforce effective stimulation patterns.

The AI-powered controller may incorporate algorithms for dynamic adjustment of neuromodulation parameters based on real-time EEG and EMG feedback. These algorithms may be implemented within the stimulation control unit 217 or the signal modulation unit 735. The system may analyze incoming EEG signals from the electrode array 630 or the strip electrode 910 to assess overall brain activity patterns. Simultaneously, EMG signals may be collected from specialized sensors to monitor muscle activity related to the targeted neurological condition.

In some cases, the AI algorithms may process the EEG and EMG data in real-time to make rapid adjustments to stimulation parameters. For example, if the EEG signals indicate an increase in abnormal neural oscillations associated with a particular symptom, the system may automatically adjust the stimulation frequency or amplitude delivered through the contact points 1025 or the coated electrodes 1030. Similarly, if the EMG signals indicate excessive muscle tension or involuntary movements, the stimulation parameters may be modified to alleviate these symptoms.

The machine learning algorithms implemented within the AI-powered controller may utilize various techniques such as deep learning, support vector machines, or decision trees to analyze the complex neural data streams. These algorithms may be trained on large datasets of patient-specific neurological data to personalize the stimulation therapy. As the system collects more data from an individual patient, the algorithms may continuously refine their models to improve the accuracy of predictions and the effectiveness of stimulation adjustments.

The AI-powered controller may also incorporate predictive modeling capabilities. By analyzing patterns in the patient's neural activity over time, the system may anticipate changes in brain state or the onset of symptoms. This predictive capability may allow the brain stimulation device 100 to proactively adjust stimulation parameters, potentially preventing or mitigating neurological events before they fully manifest.

In some implementations, the AI algorithms may utilize multi-modal data integration, combining information from various sensors and electrodes within the brain stimulation device 100. This approach may allow for a more comprehensive understanding of the patient's neurological state and may enable more nuanced and effective stimulation strategies.

The AI-powered controller may also include safety features to prevent overstimulation or unintended neural effects. These features may include built-in limits on stimulation parameters, continuous monitoring for adverse neural responses, and fail-safe mechanisms to halt stimulation if unexpected patterns are detected. The self-learning algorithms may incorporate these safety constraints into their optimization processes, ensuring that the adaptive stimulation remains within safe and effective ranges.

The brain stimulation device 100 may include a wireless interface 223 for enabling remote monitoring and control of the stimulation system. In some cases, the wireless interface 223 may facilitate data transmission between the brain stimulation device 100 and external devices.

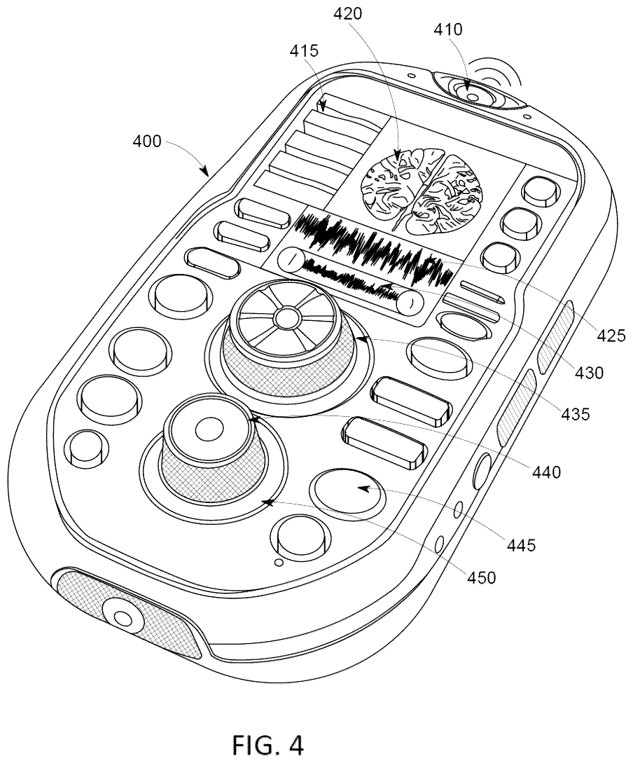

As shown in FIG. 4, a remote-control unit 400 may be used to adjust parameters of the brain stimulation device 100 shown in FIG. 1. The remote-control unit 400 may include a wireless indicator 410 to display connectivity status. A neural display 415 on the remote-control unit 400 may provide visual feedback of brain activity data. The remote-control unit 400 may also feature a waveform monitor 420 for displaying real-time neural signal patterns.

The remote-control unit 400 may incorporate a data transmission indicator 425 to show the status of communication with the brain stimulation device 100 shown in FIG. 1. A stimulation control 430 on the remote-control unit 400 may allow for adjustment of various stimulation parameters. The remote-control unit 400 may include a control dial 435 for precise parameter adjustments.

In some implementations, the remote-control unit 400 may feature a mode selector 440 for switching between different operational modes of the stimulation system. A Bluetooth button 445 on the remote-control unit 400 may facilitate wireless connectivity with the brain stimulation device 100 shown in FIG. 1. The remote-control unit 400 may also include a navigation control 450 for menu navigation and parameter selection.

FIG. 5 illustrates that the brain stimulation device 100 may incorporate a wireless receiver 555 for enabling remote communication capabilities. The wireless receiver 555 may work in conjunction with the processing chip 535 and microcontroller 550 to manage wireless data exchange and remote-control functions.

The brain stimulation device 100 may utilize a cloud-based data analytics system for predicting neurological conditions and optimizing stimulation therapy. This system may analyze data transmitted through the wireless interface 223 shown in FIG. 2 to identify patterns or trends in neural activity that could inform treatment decisions.

Electric Brain Stimulation device 100, approximately the size of a quarter, showcasing its compact and advanced design. The Electric Brain Stimulation device 100 is placed on the brain 510. The Electric Brain Stimulation device 100 has a Biocompatible outer shell 515, Precision Tuning Dial 520, Neural Interface Connector 525, High frequency data transmission port 530, AI processing chip 535, with embedded memory unit, microcontroller unit 550 and wireless receiver 555.

FIG. 8 depicts a smartphone device 810 that may be used as an interface for monitoring and controlling the brain stimulation system. The smartphone device 810 may include a neural activity display 815 for showing real-time brain activity patterns. A signal processing interface 820 on the smartphone device 810 may provide visual representation of processed neural signals.

The smartphone device 810 may incorporate a wireless interface 825 for maintaining connectivity with the brain stimulation device 100. A stimulation control 830 on the smartphone device 810 may allow for adjustment of stimulation parameters. The smartphone device 810 may also include a signal modulation unit 835 for managing the modulation of neural signals.

In some cases, the smartphone device 810 may feature a brainwave monitor 840 for displaying ongoing brain activity measurements. The smart phone or table device 810 may include stimulation settings 845 that can be adjusted through the touchscreen interface. A connectivity port 850 on the smartphone device 810 may enable both data transfer and power connection.

The wireless remote-control system, which may include the remote-control unit 400 and the smartphone device 810, may allow for external modulation of stimulation parameters. This capability may enable real-time physician intervention and adjustment of the brain stimulation device 100 without the need for direct physical access to the implanted device.

The cloud-based data analytics system may process large datasets to identify patterns in neural activity that could inform treatment protocols. By analyzing aggregated patient data, the system may potentially improve the effectiveness of stimulation therapy over time. The wireless connectivity may allow for continuous monitoring and adjustment of stimulation parameters, potentially enhancing the personalization and responsiveness of the treatment.

The brain stimulation device 100 may incorporate biofeedback integration and closed-loop control mechanisms to enhance stimulation efficiency and minimize side effects. In some cases, the brain stimulation device 100 may utilize a sensor array 125 for continuous monitoring of neural activity. The sensor array 125 may be positioned around the periphery of the brain stimulation device 100, as shown in FIG. 1.

The sensor array 125 may work in conjunction with the linear electrode array 105 to collect real-time neurophysiological data. This data may be processed by the interface circuit board 110, which may serve as the central processing unit for the closed-loop control system. In some implementations, the interface circuit board 110 may analyze the incoming neural signals to assess the effectiveness of the current stimulation parameters.

As illustrated in FIG. 2, the brain stimulation device 100 may include a stimulation control unit 217 and a signal processor 227. These components may work together to implement the closed-loop control mechanism. The signal processor 227 may analyze the neural activity data collected by the sensor array 125 and the contact electrodes 231. Based on this analysis, the stimulation control unit 217 may dynamically adjust the stimulation parameters delivered through the electrode lead 210.

In some cases, the closed-loop control system may utilize the central hub 610 and the electrode array 630 shown in FIG. 6. The central hub 610 may house the signal processing unit 645, which may be responsible for real-time analysis of neural signals. The electrode array 630 may provide multiple stimulation points, allowing for precise adjustment of stimulation patterns based on the processed feedback.

The brain stimulation device 100 may incorporate a neural interface 635 to enable direct interaction with brain tissue. The neural interface 635 may facilitate both the collection of neural activity data and the delivery of adjusted stimulation parameters. This bidirectional communication may be crucial for maintaining an effective closed-loop control system.

FIG. 6 shows the central processing hub 610, electrode coating rings and contacts 615, data transmission circuitry 620, flexible biocompatible polymer contacts 625, electrode lead array 630, high precision neural interface 635, Bio-inert coated electrodes 640 and AI-controlled signal processing unit 645.

As disclosed in the description Advanced Coatings mean:

Description: These coatings are typically composed of biocompatible materials like parylene-C, iridium oxide (IrOx), or platinum black, which enhance electrical conductivity while reducing immune response.
a. Function: They help reduce inflammation, minimize scar tissue formation, and improve signal stability over time. Some coatings also provide hydrophobic or hydrophilic properties to optimize electrode-tissue interaction.
b. Location in the Image: Visible as the shiny or textured layers on the contact rings surrounding the electrodes.

As disclosed in the description Flexible Designs:
a. Description: The leads and electrode structures are made from polymers like polyimide, PDMS (Polydimethylsiloxane), or liquid crystal polymer (LCP) to ensure flexibility while maintaining structural integrity.
b. Function: Flexibility is critical in reducing mechanical stress on brain tissue, allowing the device to move naturally with physiological movements while maintaining electrode contact.
c. Location in the Image: The thin, flexible wiring connecting the electrodes to the stimulation device showcases the design's adaptability.

As disclosed in the description High-Precision Contact Electrodes:
a. Description: The electrodes are usually constructed from platinum-iridium (Pt—Ir), gold, or conductive carbon nanotube composites to ensure high signal fidelity. They can have micro-patterned surfaces or be nano-textured to increase surface area, improving charge transfer efficiency.
b. Function: These high-precision electrodes allow for targeted neural stimulation with minimal noise, ensuring precise neuromodulation without excessive current leakage.
c. Location in the Image: The small, round contact points arranged in a ring on the device show where these high-precision electrodes are placed for optimal neural interfacing.

FIG. 7 depicts a signal modulation unit 735 that may work in tandem with the processing hub 715 to implement the biofeedback-responsive stimulation adjustments. The signal modulation unit 735 may receive processed neural activity data from the processing hub and use this information to fine-tune the stimulation parameters delivered through the contact points 730.

As shown in FIG. 7, the brain stimulation device 100 may feature a circular disc-shaped contact surface with a distinctive radial pattern design. The segmented sections may be coated with platinum-iridium for enhanced conductivity and biocompatibility. The electrode may be mounted on a flexible polymer substrate to allow for optimal conformity to brain tissue surfaces.

The brain stimulation device 100 shown in FIG. 1 may have as shown in FIG. 7 high density electrode arrays 710, Central processing hub 715, Biocompatible transparent housing 720, Flexible conductive pathways 725, platinum-iridium coated contacts 730, AI-optimized signal modulation unit 735, dynamic neural stimulation interface 740 and an integrated power and data cable 745.

To further enhance biocompatibility and reduce inflammatory responses, the electrodes may incorporate nano-coatings. As illustrated in FIG. 9, the electrode may feature multiple layers, including a flexible polymeric layer and a biocompatible layer. The nano-coatings applied to the surface may enhance conductivity while minimizing tissue reaction.

In some implementations, the closed-loop control system may be monitored and adjusted through external devices. As shown in FIG. 8, a smart phone or tablet device 810 may be used to display real-time neural activity on the neural activity display 815. The signal processing interface 820 may provide visual representation of the processed neural signals, allowing clinicians to observe the effectiveness of the closed-loop control system.

The smart phone or table device 810 may also include a stimulation control 830 and a signal modulation unit 835, which may allow for manual adjustments to the closed-loop parameters if necessary. The brainwave monitor 840 may display ongoing brain activity measurements, providing additional context for the automated stimulation adjustments made by the brain stimulation device 100.

The closed-loop control system may continuously assess the patient's response to stimulation and make real-time adjustments to optimize therapeutic outcomes. For example, if the sensor array 125 detects an increase in abnormal neural oscillations, the stimulation control unit 217 may automatically adjust the stimulation frequency or amplitude delivered through the electrode lead 210. Similarly, if the neural interface 635 indicates a reduction in symptom-related neural activity, the signal processing unit 645 may modify the stimulation parameters to maintain the therapeutic effect while minimizing unnecessary stimulation.

In some cases, the closed-loop control system may incorporate machine learning algorithms implemented within the processing chip 535 or the microcontroller 550. These algorithms may analyze patterns in the patient's neural responses over time, potentially identifying optimal stimulation strategies for individual patients or specific symptom profiles.

The biofeedback-responsive closed-loop control system may operate on multiple timescales. Rapid adjustments may be made in response to immediate changes in neural activity, while longer-term trends may be analyzed to optimize stimulation parameters over extended periods. This multi-scale approach may allow the brain stimulation device 100 to adapt to both acute symptom fluctuations and gradual changes in the patient's neurological condition.

By integrating biofeedback mechanisms and implementing closed-loop control, the brain stimulation device 100 may potentially achieve more precise and personalized neuromodulation. This approach may allow for dynamic adaptation to the patient's changing neural state, potentially improving therapeutic outcomes while minimizing unwanted side effects.

The brain stimulation device 100 may incorporate an implantable power system designed for long-term functionality and energy efficiency. In some cases, the power system may utilize inductive charging technology to enable wireless power transfer to the implanted device.

As shown in FIG. 1, the brain stimulation device 100 may include a power coil 175 integrated into the circular structure. The power coil 175 may be part of an inductive charging mechanism that allows for wireless power transfer to the implanted device. In some implementations, an external charging coil may be placed near the skin surface above the implanted brain stimulation device 100. The external coil may generate an electromagnetic field that induces a current in the power coil 175 within the brain stimulation device 100, enabling power transfer without direct electrical connections.

The power system may also incorporate energy harvesting capabilities to supplement the primary power source. Energy harvesting techniques may capture and convert ambient energy from the body or surrounding environment into usable electrical power for the brain stimulation device 100. In some cases, the energy harvesting components may work in conjunction with the power coil 175 to maximize energy efficiency and extend the operational life of the device between charging sessions.

FIG. 2 illustrates the internal components of the brain stimulation device 100, including the stimulation control unit 217 and the signal processor 227. These components may be designed to operate efficiently within the constraints of the implanted environment. The power system may incorporate advanced power management algorithms implemented within the stimulation control unit 217 or the signal processor 227 to optimize energy usage and extend the operational life of the device.

As depicted in FIG. 5, the brain stimulation device 100 may include a data transmission port 530 that enables high-frequency data communication. In some implementations, the data transmission port 530 may be designed to minimize power consumption during data transfer operations, contributing to the overall energy efficiency of the device.

The processing chip 535 and microcontroller 550 shown in FIG. 5 may be optimized for low power consumption while maintaining the computational capabilities required for AI-driven algorithms and adaptive stimulation control. In some cases, the processing chip 535 and microcontroller 550 may incorporate power-saving modes that adjust their performance based on the current operational requirements of the brain stimulation device 100.

The wireless receiver 555 illustrated in FIG. 5 may be designed to operate with minimal power draw, allowing for efficient remote communication and control of the brain stimulation device 100. In some implementations, the wireless receiver 555 may utilize low-power communication protocols to further enhance energy efficiency.

The combination of inductive charging, energy harvesting, and power-efficient components may contribute to the long-term functionality of the brain stimulation device 100. This integrated power management approach may help address challenges associated with long-term implantation, such as the need for frequent battery replacements or invasive procedures to maintain power supply. By optimizing power usage and incorporating multiple charging methods, the brain stimulation device 100 may potentially achieve extended operational periods between charging sessions, enhancing its practicality for long-term therapeutic use.

The brain stimulation device 100 may integrate multiple components to provide AI-enhanced adaptive neuromodulation therapy. As shown in FIG. 1, the brain stimulation device 100 may incorporate a linear electrode array 105, an interface circuit board 110, and a sensor array 125 arranged in a circular configuration. These components may work together to enable targeted stimulation and real-time monitoring of neural activity.

In some cases, the brain stimulation device 100 may include an electrode housing 219, as illustrated in FIG. 2. The electrode housing 219 may encase the internal components of the brain stimulation device 100, providing protection and structural support. The electrode lead 210 may extend from the electrode housing 219 to interface with brain tissue 510, allowing for direct neural stimulation and signal recording.

The stimulation control unit 217 and signal processor 227, shown in FIG. 2, may work in conjunction with the interface circuit board 110 to process neural signals and adjust stimulation parameters in real-time. The wireless interface 223 may enable remote monitoring and control of the brain stimulation device 100, facilitating personalized therapy management.

FIG. 5 depicts additional components that may contribute to the overall functionality of the brain stimulation device 100. The processing chip 535 and microcontroller 550 may handle complex computations required for AI-driven algorithms and adaptive stimulation control. The wireless receiver 555 may allow for efficient remote communication, while the data transmission port 530 may enable high-frequency data exchange with external devices.

The brain stimulation device 100 may incorporate a dual-mode stimulation protocol, allowing for automatic switching between electrical and magnetic stimulation. In some implementations, the stimulation control unit 217 may manage this dual-mode capability, adjusting the stimulation type based on real-time neural feedback and therapeutic requirements. The electrical stimulation may be delivered through the contact electrodes 231, while magnetic stimulation may be generated by specialized coils integrated within the brain stimulation device 100.

The system's functionality may be monitored and controlled through external devices. As shown in FIG. 8, a smartphone device 810 may serve as an interface for the brain stimulation system. The neural activity display 815 and signal processing interface 820 may provide visual feedback on ongoing brain activity and stimulation effects. The stimulation control 830 and signal modulation unit 835 may allow for remote adjustment of stimulation parameters, enabling personalized therapy management.

In some cases, the brain stimulation device 100 may utilize various electrode configurations to address different neurological and psychiatric disorders. The gamma electrode array 304, dental electrode array 306, strip electrode array 308, and multi-contact array 309 may provide options for targeting specific neural circuits. The circular electrode array 310, deep brain electrode 311, and matrix electrode array 315 may offer additional stimulation patterns for diverse therapeutic applications.

The integration of AI algorithms within the processing chip 535 may enable adaptive neuromodulation based on individual patient responses. The system may analyze neural activity patterns detected by the sensor array 125 and adjust stimulation parameters delivered through the electrode array 630 to optimize therapeutic outcomes. This personalized approach may potentially improve treatment efficacy for conditions such as epilepsy, Parkinson's disease, depression, and chronic pain disorders.

The closed-loop functionality of the brain stimulation device 100 may be enhanced by the continuous monitoring capabilities of the sensor array 125 and the real-time processing performed by the signal processor 227. This may allow for rapid adjustments to stimulation parameters in response to changes in neural activity, potentially preventing or mitigating adverse events such as seizures or motor symptoms.

In some implementations, the brain stimulation device 100 may leverage cloud-based data analytics to further refine its therapeutic approach. Data collected through the wireless interface 223 may be analyzed to identify long-term trends and optimize treatment protocols. This may enable the system to adapt to gradual changes in the patient's condition and potentially improve outcomes over extended periods of use.

The combination of advanced hardware components, AI-driven algorithms, and adaptive stimulation protocols may position the brain stimulation device 100 as a versatile platform for treating a wide range of neurological and psychiatric disorders. By integrating real-time neural monitoring, personalized stimulation adjustments, and remote management capabilities, the system may offer potential improvements in the efficacy and accessibility of neuromodulation therapies.

A number of implementations have been described. Nevertheless, it will be understood that various modifications may be made without departing from the spirit and scope of the disclosure. Accordingly, other implementations are within the scope of the following claims.

The controller used in the brain stimulation device 100 may employ adaptive AI algorithms that enable continuous refinement of stimulation protocols based on observed neural responses. In some cases, these algorithms may incorporate predictive models to anticipate changes in neural activity and preemptively adjust stimulation parameters to maintain optimal therapeutic effects.

The neuromodulation controller may also integrate with cloud-based computing resources to leverage more complex AI models and access larger datasets for improved decision-making. This connectivity may allow for ongoing refinement of the controller's algorithms based on aggregated data from multiple patients, potentially leading to more effective and personalized treatment protocols over time.

In some implementations, the controller may incorporate multiple processing channels to simultaneously manage different stimulation targets or modalities. This multi-channel capability may enable coordinated stimulation across various brain regions, potentially addressing complex neurological conditions that involve multiple neural circuits.

The AI-powered controller may also include safety features to prevent overstimulation or unintended neural effects. These may include built-in limits on stimulation parameters, continuous monitoring for adverse neural responses, and fail-safe mechanisms to halt stimulation if unexpected patterns are detected.

By integrating advanced AI capabilities with precise control over stimulation parameters, the neuromodulation controller may offer a sophisticated approach to adaptive brain stimulation. This system may potentially improve treatment outcomes across a range of neurological and psychiatric disorders by providing highly personalized and responsive neuromodulation therapy.

The brain stimulation system may incorporate a wireless interface for monitoring and programming stimulation parameters. This wireless interface may enable remote control and data transmission, enhancing the flexibility and functionality of the system.

As illustrated in FIG. 4, the system may include a remote-control device designed for adjusting parameters of the Electric Brain Stimulation device. The remote control may feature a sleek, ergonomic design with a display screen positioned in the upper portion. This display may show brain imaging data and waveform patterns, allowing for real-time neural activity monitoring.

The remote control may comprise multiple function buttons and rotary dials for precise parameter adjustments. In some cases, these controls may allow clinicians or patients to modify stimulation settings wirelessly, without the need for direct physical access to the implanted device.

The wireless interface may integrate Bluetooth and Wi-Fi-enabled communication capabilities. This integration may facilitate real-time data logging and remote programming of the stimulation device. The Bluetooth connectivity may be particularly useful for short-range communication, such as between the remote control and the implanted device.

In some implementations, the system may utilize cloud-based data analytics for predictive analytics and therapy optimization. As shown in FIG. 8, a smartphone interface may display real-time neuromodulation data through multiple visualization methods. This interface may connect to cloud-based services, allowing for more complex data analysis and storage.

The cloud-based system may analyze patient data to provide clinicians with predictive analytics for therapy optimization. In some cases, this may involve processing large datasets to identify patterns or trends in neural activity that could inform treatment decisions.

The wireless connectivity and cloud-based analytics may enable continuous monitoring and adjustment of stimulation parameters. This capability may allow for more responsive and personalized treatment protocols, potentially improving therapeutic outcomes for patients with various neurological disorders.

In some implementations, the wireless interface may also facilitate software updates to the implanted device, ensuring that the system remains up to date with the latest algorithms and safety features. This capability may extend the functional lifespan of the implanted device and allow for ongoing improvements in its performance.

The combination of wireless connectivity and cloud-based analytics may create a comprehensive ecosystem for managing brain stimulation therapy. This system may enable real-time monitoring, remote adjustments, and data-driven optimization of treatment protocols, potentially enhancing the effectiveness and accessibility of neuromodulation therapies.

The brain stimulation system may incorporate biofeedback integration and closed-loop control mechanisms to enhance stimulation efficiency and minimize side effects. This approach may allow for real-time adaptation of stimulation parameters based on the patient's neural activity and physiological responses.

In some cases, the system may include embedded electromyography (EMG) and electroencephalography (EEG) sensors for continuous monitoring of brain function and muscle activity. These sensors may provide real-time data on neural oscillations, muscle contractions, and other relevant physiological signals.

The EMG sensors may detect electrical activity produced by skeletal muscles, which may be particularly useful in monitoring motor symptoms associated with various neurological disorders. The EEG sensors, on the other hand, may record the brain's electrical activity, providing insights into overall brain function and specific neural patterns.

As illustrated in FIG. 8, the system may incorporate a user interface displaying real-time neuromodulation data. This interface may present various visualizations of neural activity, including waveform displays and frequency graphs. The stimulation frequency and other parameters may be dynamically adjusted based on the incoming biofeedback data.

The closed-loop control system may utilize the data from these sensors to continuously assess the effectiveness of the current stimulation parameters. In some implementations, the system may employ advanced algorithms to analyze the incoming data and make real-time adjustments to the stimulation settings.

For example, if the EEG sensors detect an increase in abnormal neural oscillations associated with a particular symptom, the system may automatically adjust the stimulation frequency or amplitude to counteract this activity. Similarly, if the EMG sensors indicate excessive muscle tension or involuntary movements, the stimulation parameters may be modified to alleviate these symptoms.

The closed-loop control mechanism may operate on multiple timescales. In some cases, rapid adjustments may be made in response to immediate changes in neural activity or muscle responses. In other cases, the system may analyze longer-term trends to optimize stimulation parameters over extended periods.

By integrating biofeedback and implementing closed-loop control, the brain stimulation system may potentially achieve more precise and personalized neuromodulation. This approach may allow for dynamic adaptation to the patient's changing neural state, potentially improving therapeutic outcomes while minimizing unwanted side effects.

The system may also incorporate safeguards within the closed-loop control mechanism to prevent overstimulation or rapid fluctuations in stimulation parameters that could cause discomfort or adverse effects. These safeguards may include predefined limits on parameter changes and gradual adjustment protocols to ensure smooth transitions in stimulation settings.

In some implementations, the biofeedback integration and closed-loop control may be complemented by machine learning algorithms. These algorithms may analyze patterns in the patient's neural and muscular responses over time, potentially identifying optimal stimulation strategies for individual patients or specific symptom profiles.

The combination of real-time biofeedback, closed-loop control, and advanced data analysis may create a highly responsive and adaptive brain stimulation system. This approach may offer potential advantages in treating a wide range of neurological and psychiatric disorders by providing personalized, dynamic neuromodulation tailored to each patient's unique neural activity patterns and physiological responses.

The brain stimulation system may incorporate an implantable power system designed for long-term functionality and energy efficiency. In some cases, this power system may utilize inductive charging technology to enable wireless power transfer to the implanted device.

The inductive charging mechanism may involve an external charging coil placed near the skin surface above the implanted device. This external coil may generate an electromagnetic field that induces a current in a receiving coil within the implanted device, allowing for power transfer without direct electrical connections.

In some implementations, the system may also incorporate energy harvesting capabilities to supplement the primary power source. Energy harvesting techniques may capture and convert ambient energy from the body or surrounding environment into usable electrical power for the device.

The assembly of all the items into a kit makes the installation simple and easy for the user because the parts necessary to brain stimulation device 100 to the patient's brain and provide the required stimulation.

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI), or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information, or request that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments; it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A brain stimulation system, comprising:
an implantable electrode assembly configured to interface with neural tissue;
an artificial intelligence (AI) powered controller operatively connected to the electrode assembly and configured to dynamically adjust stimulation parameters based on real-time neurophysiological feedback; and
a wireless interface configured to enable remote monitoring and programming of the stimulation parameters.

2. The brain stimulation system of claim 1, wherein the electrode assembly comprises a flexible polymer substrate with multiple electrode contacts arranged in a radial pattern.

3. The brain stimulation system of claim 2, wherein the electrode contacts are coated with a biocompatible nano-coating to enhance conductivity and reduce inflammatory responses.

4. The brain stimulation system of claim 1, wherein the AI-powered controller utilizes a machine learning model trained on patient-specific neurological data to personalize stimulation therapy.

5. The brain stimulation system of claim 4, wherein the AI-powered controller implements a real-time seizure detection algorithm to predict and mitigate seizure events.

6. The brain stimulation system of claim 1, further comprising a cloud-based data analytics system for analyzing aggregated patient data and providing predictive analytics for therapy optimization.

7. The brain stimulation system of claim 6, wherein the cloud-based data analytics system enables remote patient monitoring and physician-controlled parameter adjustments.

8. A method of brain stimulation therapy, comprising:
receiving, by an artificial intelligence (AI) powered controller, real-time neurophysiological feedback from an implantable electrode assembly interfacing with neural tissue;
dynamically adjusting, by the AI-powered controller, stimulation parameters based on the real-time neurophysiological feedback; and
delivering stimulation to the neural tissue through the implantable electrode assembly using the adjusted stimulation parameters.

9. The method of claim 8, wherein the real-time neurophysiological feedback comprises electroencephalographic (EEG) and electromyographic (EMG) data.

10. The method of claim 9, wherein dynamically adjusting the stimulation parameters comprises modifying at least one of stimulation frequency, pulse width, and amplitude based on the EEG and EMG data.

11. The method of claim 8, further comprising implementing a closed-loop control mechanism to continuously modify stimulation based on detected brainwave activity.

12. The method of claim 11, wherein the closed-loop control mechanism utilizes a machine learning algorithm to analyze patterns in neural responses over time.

13. The method of claim 8, further comprising transmitting aggregated patient data to a cloud-based analytics system for long-term therapy optimization.

14. The method of claim 13, wherein the cloud-based analytics system provides predictive analytics for adjusting stimulation parameters based on historical patient data and population-level trends.

15. A wearable or implantable wireless neurostimulation device, comprising:
an electrode assembly configured to interface with neural tissue;
a stimulation control unit configured to deliver stimulation to the neural tissue through the electrode assembly;
a sensor array configured to detect neural activity; and
a wireless interface configured to transmit neural activity data to a cloud-based data analytics system for remote patient monitoring and predictive analytics.

16. The device of claim 15, wherein the electrode assembly comprises a flexible polymer substrate with multiple electrode contacts arranged in a radial pattern.

17. The device of claim 16, wherein the electrode contacts are coated with a biocompatible nano-coating to enhance conductivity and reduce inflammatory responses.

18. The device of claim 15, wherein the stimulation control unit comprises an artificial intelligence (AI) powered controller configured to dynamically adjust stimulation parameters based on the detected neural activity.

19. The device of claim 18, wherein the AI-powered controller implements a real-time seizure detection algorithm to predict and mitigate seizure events.

20. The device of claim 19, wherein the cloud-based data analytics system provides predictive analytics for adjusting stimulation parameters based on historical patient data and population-level trends.
